(19) United States
(12) Patent Application Publication
(10) Pub. No.: US 2004/0214756 A1
(43) Pub. Date: Oct. 28, 2004

(54) PHARMACEUTICALS FOR TREATING SEPSIS AND SEPTIC SHOCK

(75) Inventors: GERHARD DICKNEITE, MARBURG (DE); KLAUS BOSSLET, MARBURG (DE)

Correspondence Address: FINNEGAN, HENDERSON, FARABOW, GARRETT & DUNNER, LLP, 1300 I STREET, NW, WASHINGTON, DC, 20005, US

(73) Assignee: AVENTIS BEHRING GmbH

(21) Appl. No.: 08/789,406

(22) Filed: Jan. 29, 1997

Related U.S. Application Data
(63) Continuation of application No. 08/247,549, filed on May 23, 1994, now abandoned.

(30) Foreign Application Priority Data
May 25, 1993 (DE) P 43 17 282.2

(51) Int. Cl. A61K 38/17

(52) U.S. Cl. 514/12

(57) ABSTRACT

The invention relates to a two-component system for the treatment and prophylaxis of sepsis and of septic shock, where the components are intended to act in combination with each other, to a pharmaceutical and a packaging unit containing both the components, and to a process for their preparation.

The invention relates to a two-component system for the treatment and prophylaxis of sepsis and septic shock, where the components are intended to act in combination with each other, to a pharmaceutical and to a packaging unit containing both components, and to a process for their preparation.

Despite the advances which have been made in antibiotic therapy, bacterial sepsis is a central problem in intensive care, and mortality in septic shock is, at 50-70%, still unacceptably high.

It is particularly Gram-negative bacteria which have been demonstrated in patients suffering from sepsis; potential sources of infection are the gastrointestinal tract, the deferent urinary tracts and the respiratory tract, and also infected wounds and burns.

Following liberation of endotoxins (lipopolysaccharide, LPS) from the cell walls of Gram-negative bacteria, the LPS binds to the lipopolysaccharide-binding protein (LPB), and-the LPS/LPB complex binds to macrophages via the CD14 receptor. The macrophages which have been stimulated in this way secrete cytokines such as TNFα, interleukin 1 and interleukin 6, interalia. Granulocytes are activated by these mediators, and the endothelium is converted from an anticoagulatory condition into a procoagulatory condition. As a result of the expression of thromboplastin, the extrinsic pathway of coagulation is activated by the formation of a factor VII/thromboplastin complex. This results in activation of the prothrombinase complex and consequent conversion of inactive prothrombin into enzymatically active thrombin (factor IIa). The cleavage of fibrinogen into fibrin results in the formation of microthrombi (disseminated intravascular coagulopathy, DIC) in the terminal vascular bed. The decreased blood flow through the bed leads to a deficiency in oxygen supply and, as a consequence, to organ dysfunction (multiple organ failure). On the other hand, LPS can activate factor XII (Hagemann factor) directly by contact activation of the intrinsic coagulation system, and thereby also activate the kallikrein/kinin system and the complement system. The formation of bradykinin causes a fall in blood pressure, and thereby also contributes to the development of septic shock.

It is known, for the purpose of sepsis therapy, to employ antibodies against LPS, as well as antibodies, antagonists or soluble receptors against TNF or interleukin 1.

The formation of microthrombi can be suppressed by inactivating thrombin, which is the central protease of the coagulation system.

Antithrombin III is the physiological inhibitor of thrombin, and antithrombin III protein, having a molecular weight of 58 kD, can be isolated from human plasma. Antithrombin III reacts with thrombin in a stoichiometric ratio, the rate of the reaction being greatly increased by sulfated polysaccharides, especially heparin.

A marked drop in the plasma level of active antithrombin III is observed in sepsis and in septic shock, suggesting, on the one hand, increased consumption (formation of a thrombin-antithrombin complex) and, on the other, proteolytic degradation by serine proteases, especially by elastase, which is secreted from polymorphonuclear granulocytes.

It is known that antithrombin III replacement can be employed in sepsis (B. Blauhut et al., Thromb. Res. 39, 81-89, 1985).

Hirudin represents another, highly specific, inhibitor of thrombin. Hirudin has also been demonstrated to possess activity in experimental sepsis (H. Hoffmann et al., Am. Rev. Respir, Dis. 142, 782-788, 1990).

It was found that both the compounds reduced the mortality rate due to sepsis and prolonged survival time.

It has now been found, surprisingly, that the antithrombotic therapy of sepsis can be improved by combining it with substances which do not act on the coagulation system, and that this combination leads to a further reduction in mortality in sepsis and in septic shock.

Examples of suitable thrombin inhibitors are antithrombin III (AT III) prepared from human plasma or recombinantly, and mutants thereof possessing thrombin-inhibiting activity, natural or recombinantly prepared hirudin, and mutants of hirudin possessing thrombin-inhibiting activity, or synthetic thrombin inhibitors, that is chemically prepared substances which are notable for their thrombin-inhibiting effects.

Those compounds which are suitable for combining with an antithrombotic principle are substances which influence the formation, the liberation, the plasma and tissue levels and the receptor binding of cytokines, and, in addition, inhibitors of complement and of the kallikrein/kinin system.

Suitable substances are inhibitors, antagonists or soluble receptors of cytokines or antagonists of cytokine receptors, preferably of the cytokines TNF (tumor necrosis factor) and IL-1, or are the fusion proteins of these substances with an Fc moiety of an antibody.

Examples of suitable cytokine- and receptors or antagonists of cytokines are substances which suppress the biological activity of interleukin 1, for example recombinantly prepared soluble IL-1 receptor, recombinantly prepared IL-1 receptor, or an Fc-fusion protein which contains IL-1 receptor, antagonists of IL-1, i.e. IL-1-like polypeptides which bind to the receptor which do not trigger any signal, substances which suppress the biological activity of tumor necrosis factor (TNF), for example recombinantly prepared soluble TNF receptor, recombinantly prepared TNF receptor or an Fc-fusion protein which contains TNF receptor, antagonists of TNF, i.e. TNF-like polypeptides which bind to the receptor but do not trigger any signal, or recombinantly prepared interleukin 10, or its mutants, which bind to the IL-10 receptor and trigger a signal at that site. Examples of suitable inhibitors of complement or kallikrein are C1 esterase inhibitor (C1INH), purified from human plasma or prepared recombinantly, and its mutants possessing enzyme-inhibiting activity, synthetic complement inhibitors, that is chemically prepared substances which are notable for their complement-inhibiting effect, natural or recombinantly prepared aprotinin, or its kallikrein-inhibiting mutants, or synthetic kallikrein inhibitors, that is chemically prepared substances which are notable for their kallikrein-inhibiting effect.

Combinations of antithrombin III (for example Kybernin®, Behringwerke AG) or rec. hirudin (Behringwerke AG) with C1 esterase inhibitor (for example Berinert®, Behringwerke AG) are particularly preferred.

| | | |
|---|---|---|
| Dosages: | AT III | 5-1000 U/kg |
| | rec. hirudin | 0.1-20 mg/kg |
| | C1INH | 5-1000 U/kg |

The following examples explain the invention in more detail:

### EXAMPLE 1

Female CD rats were treated with a lethal dose of endotoxin (50 mg/kg, i.v.). Three groups were formed which were infused intravenously (1 ml/h) for 5 hours, beginning 15 minutes prior to the administration of LPS. Group 1 received physiological sodium chloride solution, group 2 received 0.17 mg/kg×h recombinant hirudin, group 3 received the combination therapy of 0.17 mg/kg×h recombinant hirudin and 100 units/kg×h C1 esterase inhibitor. Table 1 shows that, while rec. hirudin elicits clear prolongation of the survival rate in comparison with the control, the combination therapy using C1 esterase inhibitor was, surprisingly, clearly superior to the therapy using the antithrombotic on its own.

TABLE 1

Mortality
(% dead)

| | 6 h | 10 h |
|---|---|---|
| 1. Control (n = 8) | 50 | 88 |
| 2. rec. hirudin, | 0 | 66 |
| 0.17 mg/kg × h (n = 9) | | |
| 3. rec. hirudin, | 0 | 20 |
| 0.17 mg/kg × h + | | |
| C1 inhibitor, | | |
| 100 U/kg × h (n = 10) | | |

Significancies:
1 −> 2 p < 0.01
1 −> 3 p < 0.01
1 −> 3 p < 0.01
2 −> 3 p < 0.05

### EXAMPLE 2

Two groups were formed using the same animal model as in Example 1: the first group received an infusion of 37.5 U/kg×h of the thrombin inhibitor antithrombin III, isolated from human plasma, and the second group additionally received 125 U/kg×h C1 esterase inhibitor. Table 2. shows that the combination therapy using antithrombin III and C1INH was superior to the monotherapy using the antithrombotic on its own.

TABLE 2

| | Mortality after 8 h |
|---|---|
| | (dead/total) |
| 1. AT III | 19/30 |
| 37.5 U/kg × h | |
| 2. AT III, 37.5 U/kg × h + | 11/30 |
| C1INH, 125 U/kg × h | |

Significance:
1 −> 2 p < 0.05

Consequently, it can be demonstrated-that antithrombotic therapy using antithrombin III and hirudin can be combined with other principles for the prophylaxis and therapy of sepsis and of septic shock.

1. A pharmaceutical composition for the treatment or prophylaxis of sepsis and septic shock, comprising as a first component a thrombin inhibitor and as a second component a substance that influences the formation, the liberation, the plasma and tissue level, or the receptor binding of cytokines, or is an inhibitor of complements or is an inhibitor of the kallikrein/kinin system, wherein said first and second components are intended to act in combination with each other, together with a pharmaceutically acceptable excipient.

2-3. (Canceled)

4. A pharmaceutical composition as claimed in claim 1, wherein the thrombin inhibitor is antithrombin III.

5. A pharmaceutical composition as claimed in claim 1, wherein the thrombin inhibitor is natural or synthetic hirudin.

6. A pharmaceutical composition as claimed in claim 1, wherein the second component is a recombinantly prepared, soluble, interleukin I receptor or receptor antagonist, or its Fc-fusion protein, a recombinantly prepared, soluble, TNF receptor or receptor antagonist, or its Fc-fusion protein, a recombinantly prepared interleukin 10, an inhibitor of complement, or a C1 esterase inhibitor.

7. A pharmaceutical composition as claimed in claim 1, wherein the second component is an inhibitor of the kallikrein/kinin system.

8. A pharmaceutical composition as claimed in claim 5, wherein the second component is aprotinin that has been isolated from mammalian tissue.

9. (Canceled)

10. A method of preparing a pharmaceutical composition for the treatment of sepsis or septic shock, which comprises the step of combining a thrombin inhibitor, a cytokine antagonist, or an inhibitor of the kallikrein/kinin system, or an inhibitor of compliment.

11. A method for the treatment of sepsis or septic shock, comprising the step of administering to a patent in need thereof a thrombin inhibitor and a substance that influences the formation, the liberation, the plasma and tissue levels, or the receptor binding of cytokines, or is an inhibitor of complement or an inhibitor of the kallikrein/kinin system.

12. A pharmaceutical composition as claimed in claim 4, where the antithrombin III has been obtained from human plasma.

13. A pharmaceutical composition as claimed in claim 4, wherein the antithrombin III is recombinant antithrombin III.

14. A pharmaceutical composition as claimed in claim 5, wherein the hirudin has been recombinantly prepared.

15. A pharmaceutical composition as claimed in claim 1, wherein the first component is antithrombin II or hirudin and the second component is an IL-I inhibitor, a TNF inhibitor, C1 estrase inhibitor or aprotinin.

16. A pharmaceutical composition as claimed in claim 17 wherein the first component is antithrombin III and the second component is C1 esterase inhibitor.

17. A pharmaceutical as claimed in claim 17 wherein the first component is hirudin and the second component is C1 esterase inhibitor.
